(19) United States
(12) Patent Application Publication
(10) Pub. No.: US 2013/0332192 A1
(43) Pub. Date: Dec. 12, 2013

(54) AMPUTATED EXTREMITY HOLDER AND SAMPLE COLLECTION DEVICE

(71) Applicant: Althea McPhail, Norcoss, GA (US)

(72) Inventor: Althea McPhail, Norcoss, GA (US)

(21) Appl. No.: 13/491,482

(22) Filed: Jun. 7, 2012

(51) Int. Cl. C12M 1/00; G06Q 50/24

(52) U.S. Cl. 705/3; 435/283.1

(57) ABSTRACT

A portable amputated extremity holder and sample collection device according to various embodiments can include a first supporting member and a second supporting member. A pair of rods is disposed so as to adjustably connect the first supporting member and the second supporting member to form an extremity holder and a sample collection zone. The extremity holder is configured to hold an amputated extremity during an examination for performing a sample collection procedure on the amputated extremity. The sample collection zone comprises a plurality of testing and measuring components associated with at least one of the supporting members to assist with the sample collection procedure.

### FIELD

The present teachings relate generally to an amputated extremity holder and sample collection device to offer laboratory professionals a compact, space-saving sample collection platform, which is designed to save valuable space in crowded work areas and to ensure maximum portability while performing sample collection on an amputated limb to gather tissue specimens for histological diagnosis. The device is manufactured to be compact and lightweight for easy storage.

### Introduction

Amputations are an increasing problem among individuals with atherosclerotic vascular disease. Many of these people have diabetes mellitus which is often associated with circulatory problems. The circulatory problems occur because the arteries that carry blood to the extremities become narrowed or clogged. This interferes with the normal flow of blood and sometimes causes numbness and pain. Every organ and tissue of the body that continuously experiences partial or complete, acute or chronic arterial restricted blood circulation is susceptible to suffer irreversible damage. Tissue damage might also occur as a consequence of chronic venous drainage failure. All these disorders are frequently found in diabetic patients. In these individuals, circulation might fail due to macro and micro vascular lesions. Furthermore, peripheral nerve structures are also affected which contributes to the circulatory deterioration. Diabetic-associated damages to the autonomic nervous system results in the failure of the limb's skin defense mechanisms such as sweating and sebaceous secretion. Local insensibility renders the extremity prone to local traumas which might evolve to a problem wound. As a result of this healing defect that is further complicated by the circulatory disturbances, many diabetic patients undergo limb amputation.

Amputation, of course, involves severing arteries and veins and care must be exercised to ensure that the correct area is totally amputated. After amputation, the amputated limb is sent to the lab where a specimen is taken to confirm that the portion of the patient's limb that includes the damaged veins and nerves is completely contained within the excised specimen. The portion of the patient's limb containing the damage veins and nerves should ideally be totally excised with a small margin or rim of normal veins, nerves and tissue on all sides.

Thus, there is a need to accurately take samples for histological evaluation. Sections from the vessels of the amputated limbs are evaluated for atherosclerosis or other vascular diseases. Sections to evaluate the viability of the specimen margins and any additional lesion are also obtained. After the evaluation, a report documenting the extent of the vascular disease is prepared. Obtaining good margins of normal tissue during the amputation is extremely important because, if the amputated limb has not been excised to include veins and nerves having proper circulation, then the amputation process must be revised and a subsequent amputation at a higher location must be repeated on the patient's remaining limb to actually obtain tissue where there is adequate blood flow and viable tissue. Highly trained personnel must perform the analysis on the amputated extremity to collect the sample for histology to accurately confirm that the good margins of normal tissue were obtained. However, the sample collection process can be completed more rapidly if an assistant to the laboratory professional holds the specimen because of the typically large size and awkward shape of the specimens. Requiring two or more personnel to conduct the sample collection can be relatively time-consuming and expensive. Thus, it may be desirable to provide a sample preparation device that could securely hold the specimen and be used by a single trained professional to facilitate the quick, efficient, and accurate performance of the sample collection.

### SUMMARY

The present invention may satisfy one or more of the above-mentioned desirable features. Other features and/or advantages may become apparent from the description which follows.

A device according to various exemplary embodiments can include a first supporting member and a second supporting member. A pair of rods may be disposed so as to adjustably connect the first supporting member and the second supporting member to form an extremity holder and a sample collection zone. The extremity holder may be configured to hold an amputated extremity during an examination for performing a sample collection procedure on the amputated extremity. The sample collection zone may comprise a plurality of testing and measuring components associated with at least one of the supporting members to assist with the sample collection procedure.

A device according to various exemplary embodiments can include a first supporting member and a second supporting member. A pair of rods may be disposed so as to adjustably connect the first supporting member and the second supporting member to form an extremity holder and a sample collection zone. The extremity holder may be configured to hold an amputated extremity during an examination for performing a sample collection procedure on the amputated extremity. The extremity holder may comprise a pair of opposed rotating holding members for securely supporting the amputated extremity without damaging the amputated extremity and may be configured to rotate the amputated extremity during the sample collection procedure. The sample collection zone may comprise a plurality of testing and measuring components associated with at least one of the supporting members to assist with the sample collection procedure.

A device according to various exemplary embodiments can include a first supporting member and a second supporting member. A pair of rods may be disposed so as to adjustably connect the first supporting member and the second supporting member to form an extremity holder and a sample collection zone. The extremity holder may be configured to hold an amputated extremity during an examination for performing a sample collection procedure on the amputated extremity. The sample collection zone may comprise a plurality of testing and measuring components associated with at least one of the supporting members to assist with the sample collection procedure. The sample collection zones may comprise at least one retractable component movable from a retracted position within a housing of at least one of the supporting member to an extended position in which the at least one retractable holder extends from the housing of the at least one of the supporting member.

A device according to various exemplary embodiments can include a first supporting member and a second supporting member. A pair of rods may be disposed so as to adjustably connect the first supporting member and the second supporting member to form an extremity holder and a sample collection zone. The extremity holder may be configured to hold an amputated extremity during the examination. The sample collection zone may comprise a plurality of testing and measuring components associated with at least one of the supporting members to assist with the sample collection procedure. A processor may be provided within the device to communicate with and coordinate operation of various sensors and components to control the device. The processor may be connected to a network. The network may be connected to interface with a patient pathology information system to generate report, store and transmit patient information related to the amputated extremity. The extremity holder may comprise a pair of opposed rotating holding members for securely supporting the amputated extremity without damaging the amputated extremity and may be configured to rotate the amputated extremity during the sample collection procedure. The sample collection zones may comprise at least one retractable component movable from a retracted position within a housing of at least one of the supporting member to an extended position in which the at least one retractable holder extends from the housing of the at least one of the supporting member. The processor may control movement of the pair of rods to adjustably connect the first and second holding members, movement of the pair of opposed rotating holding members, and movement of the at least one retractable component. A display in communication with the processor may be configured to display the patient information.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

### BRIEF DESCRIPTION OF THE DRAWINGS

The skilled artisan will understand that the drawings described below are for illustrative purposes only. The drawings are not intended to limit the scope of the present teachings in any way.

FIG. 1 is a perspective view of an exemplary embodiment of an amputated extremity holder and sample collection device according to the present teachings;

FIGS. 2-4 are side views of a pair of collapsible rods moving from an extended position to a retracted position, which can be included within the amputated extremity holder and sample collection device;

FIG. 5 is another exemplary embodiment of an amputated extremity holder and sample collection device according to the present teachings;

FIG. 6 is a top view of an exemplary embodiment of a single holder, which can be included within the amputated extremity holder and sample collection device;

FIG. 7 is a top view of an exemplary embodiment of a pair of holders, which can be included within the amputated extremity holder and sample collection device;

FIG. 8 is a perspective view of a retractable holder, which can be included within the amputated extremity holder and sample collection device;

FIGS. 9A-9F depict exemplary embodiments of containers, which can be employed with the amputated extremity holder and sample collection device;

FIGS. 10A-10B illustrate a gripper, which can be included within the amputated extremity holder and sample collection device; and

FIG. 11 is a perspective view of yet another exemplary embodiment of an amputated extremity holder and sample collection device.

### DETAILED DESCRIPTION OF VARIOUS EMBODIMENTS

Reference will now be made to various embodiments, examples of which are illustrated in the accompanying drawings. However, these various exemplary embodiments are not intended to limit the disclosure. On the contrary, the disclosure is intended to cover alternatives, modifications, and equivalents.

Throughout the application, description of various embodiments may use “comprising” language, however, it will be understood by one of skill in the art, that in some specific instances, an embodiment can alternatively be described using the language “consisting essentially of” or “consisting of”

For purposes of better understanding the present teachings and in no way limiting the scope of the teachings, it will be clear to one of skill in the art that the use of the singular includes the plural unless specifically stated otherwise. Therefore, the terms “a,” “an” and “at least one” are used interchangeably in this application. Unless otherwise indicated, all numbers expressing quantities, percentages or proportions, and other numerical values used in the specification and claims, are to be understood as being modified in all instances by the term “about.”

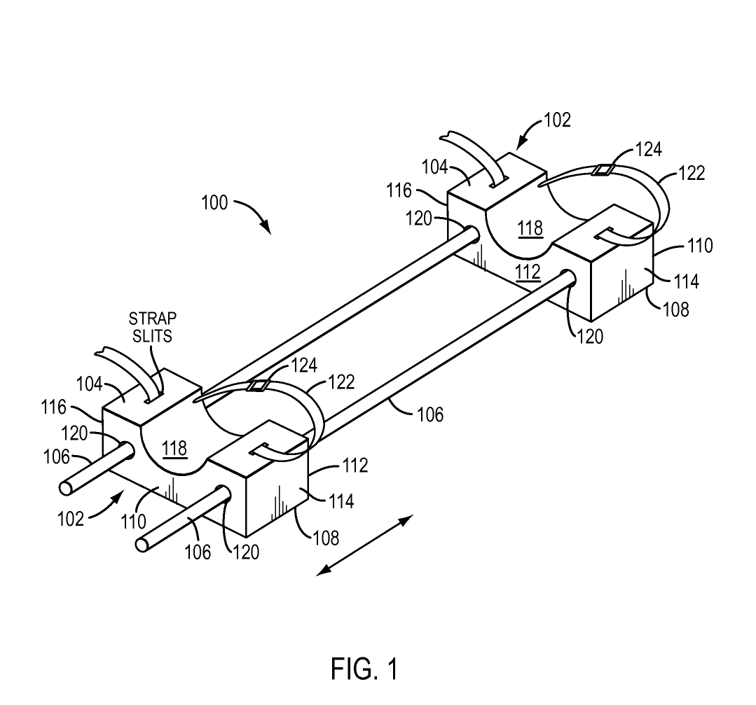

An exemplary embodiment of an amputated extremity holder and sample collection device 100 that can be used, for example, to take a sample for use in pathology and histology applications, is illustrated in FIG. 1. The device 100 can provide an extremity holder 102 and a sample collection zone 104 for performing a sample collection protocol on an amputated limb to gather tissue specimens at which pathology and histology analysis may occur.

The holder and sample collection device 100 can include a pair of opposed holding members 102 for supporting and securing an amputated extremity, such as an arm or leg. In the exemplary embodiment shown in FIG. 1, each holding member 102 is configured to resemble a box including a base 108, a front wall 110, a back wall 112 and an opposing pair of side walls 114 and 116 extending upwards from the base 108 towards a supporting surface 118. The supporting surface 118 may serve as a top surface for receiving the amputated extremity. The supporting surface 118 may be configured with any desired shape, such as a flat planar surface or a curvilinear surface. For example, in the exemplary embodiment, the supporting surface 118 is depicted as having a substantially curve supporting surface. In this embodiment, the holding member 102 is made of acrylic, but could also be made of other materials, including, but not limited to, ceramic, wood, metal, plastic, glass or any other suitable material. In some embodiments, the device 100 may include a rotating mechanism 136 to enable the laboratory professional to partially or completely rotate the extremity limb during the sample collection process, which will be further described below.

A pair of adjusting rods 106 may extend through holes 120 provided within each holding member 102 such that one holding member 102 is adapted to be positioned laterally with respect to another holding member 102. The lateral adjustability allows the pair of holding members 102 to adjust their positions to accommodate the insertions of various sized amputated extremities. The adjusting rod 106 can be made from aluminum or plastic for lightness or stainless steel if a more durable product is required. Preferably the entire device is compact and lightweight to be used by a laboratory professional at a laboratory sink in a prep area. In some embodiments, the entire device weighs approximately between 5 to 10 lbs. In some embodiments by the adjusting the rods, the length of the device can extend from 10 to 20 inches.

Straps 122 are provided for securely restraining the amputated extremity in the holding member 102 so as to prevent both longitudinal and rotational movement of the extremity with respect to the holding member 102 during the sample collection process. The straps 122 can be readily adjusted through an adjustable latching mechanism 124 to vary the tension to fit various sized and shaped extremities. The strap 122 can be made of leather, fabric, vinyl, rubber or elastomer, such as plastic, and they are mounted on each holding member 102, with the adjustable latching mechanism 124. The exemplary embodiment in FIG. 1 depicts the adjustable latching mechanism 124 as a buckle. Similarly, straps 122 can be interlocked with one or more latching mechanism such as, for example, a snap, a clasp, and VELCRO hook-and-loop, among other fasteners.

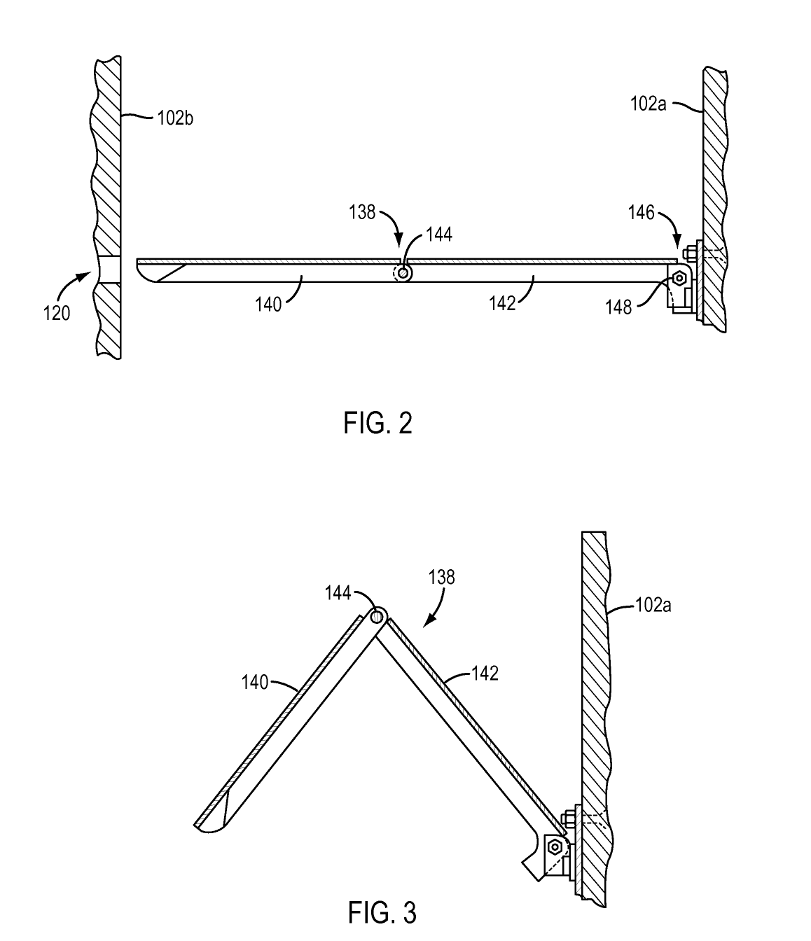

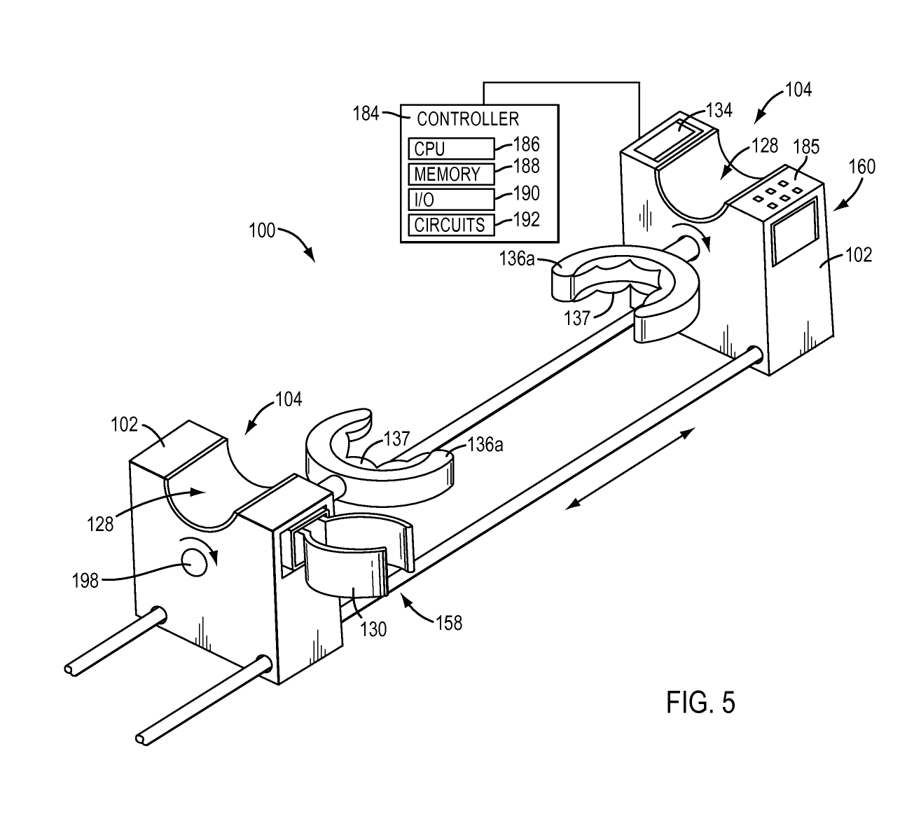

In general, the device 100 offers laboratory professionals a compact, space-saving sample collection platform, which is designed to save valuable space in crowded work areas and to ensure maximum portability while performing a sample collection on an amputated limb to gather tissue specimens for histological diagnosis. The device is manufactured to be compact and lightweight for easy storage. Some embodiments of the device may be configured to be foldable or collapsible for easy storing and transporting. FIGS. 2, 3, and 4 illustrate a side view of collapsible rods 138 going from an extended position to a collapsed and folded position. A pair of foldable arms 140, 142 is hingedly connected to one of the holding member 102a. Foldable arm 140 and 142 are pivotally joined at adjacent ends by a pin 144. In the exemplary embodiments of FIGS. 2, 3, and 4, folding arm 142 is connected to holding member 102a by a hinge arrangement. Specifically, a pivot screw 148 can be provided in order to rotatably connect folding arm 142 to holding member 102a. FIG. 3 depicts the arms in a partially collapsed position. As shown in FIG. 4, when the device is collapsed, the first foldable arm 140 and the second foldable arm 142 fold together. Once collapsed, the space requirement of the device 100 is dramatically reduced making it easy to handle, store and transport. In the embodiments having collapsible and foldable rods, the device 100 can be assembled to appear as shown in FIG. 1. For example, a pair of the first foldable arms 140 (in FIG. 6, only one of the pair of first foldable arms 140 is shown) of the collapsible rods may extend through a pair of holes 120 (in FIG. 6, only one of the pair of holes is shown) provided within holding member 102b such that holding member 102b is adapted to be positioned laterally with respect to holding member 102a. Holding member 102b can be provided with a locking mechanism (not shown) to lock holding member 102b in a secure position relative to holding member 102a.

In some embodiments, an actuator and motor (not shown) may be connected to one holding member and the rods to move the holding member along the rods relative to the other holding member. In some embodiments, the adjusting rods may be configured as a pair of telescoping rods that extends and retracts, which make the holding members move relative to each other.

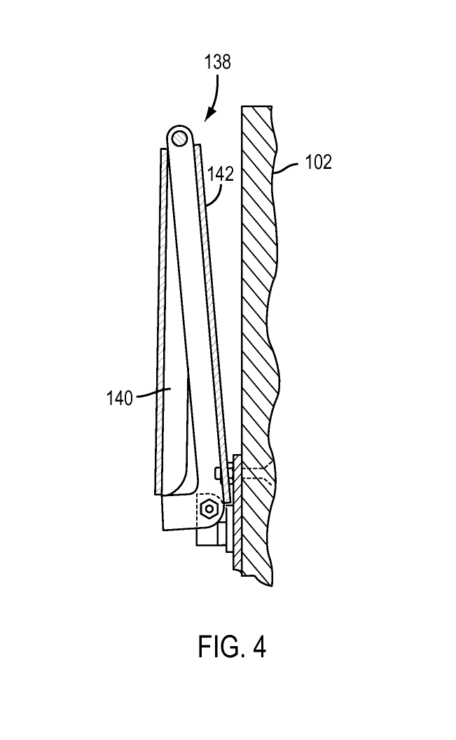

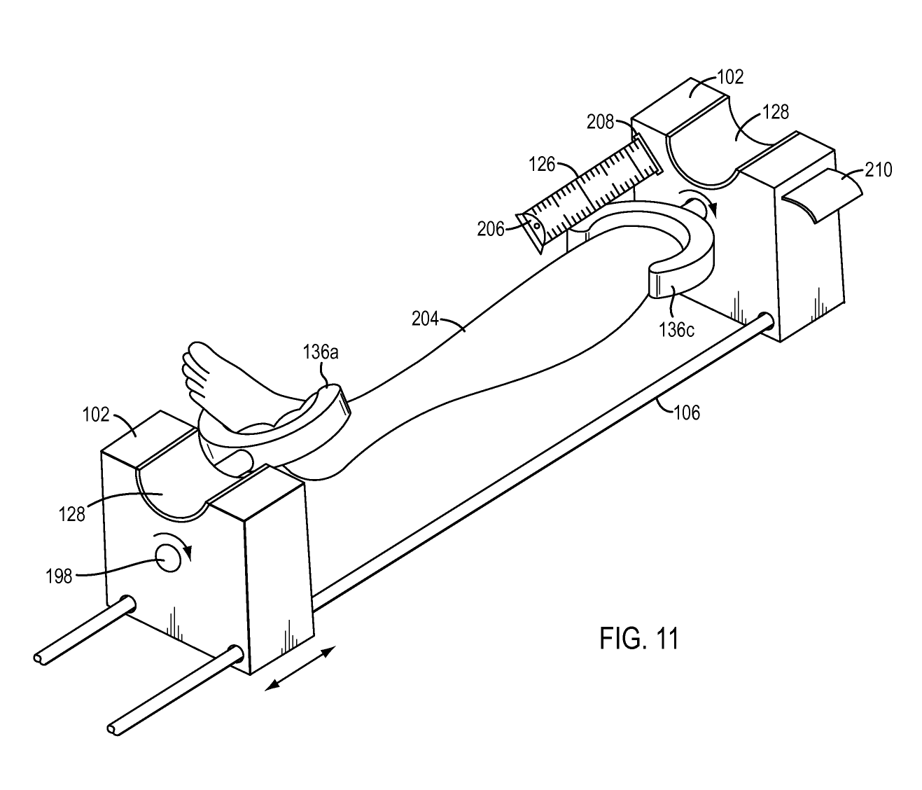

As illustrated in the exemplary embodiments of FIGS. 5-8, 10, and 11, the sample collection zone 104 may include a variety of components to assist with the sample collection and testing process. For example, one or more rulers 126 (FIG. 11) may be provided to measure the dimensions of the amputated extremity and the specimen. In some embodiments, a retractable ruler 126 may be disposed within a holding member 102 wherein a tab 206 of the ruler remains external to the holding member and the tape which contains incremental measurements retracts within the holding member 102. The tab 206 is sized larger than an opening 208 provided in the holding member to prevent the tab 206 from entering into the interior of the holding member 102 during retraction. When needed, the tab 206 of the retractable ruler 126 is pulled and the extracted through the opening 208. A cradle (not shown) may be provided within the holding member 102 to receive the tape portion when the retractable ruler 126 is not in use. Alternatively or in conjunction, in some embodiments, the adjusting rods 106 can also have measurement indicators (inches, centimeters, etc.) printed thereon to function as a ruler. As the holding members 102 are laterally adjusted, the measurement indicators can also be used to measure the length of the extremity.

In some embodiments as shown in FIG. 5, the device 100 may be configured having electronic components. A mini-computer controller or processor 184 may be provided within the device 100 to communicate with and coordinate operation of the various sensors and components included in both holding members 102 and other portions of the device 100 to permit automatic operation during specimen collection and preparation. The processor may include an appropriate operator interface with associated input keypad or buttons 185 and an output display 134, such as a liquid crystal diode display. For example, the display 134 can be connected to a scale 128 to show the weight and length of the specimen and the sample weight in real time, as well as other typical displays for a scale. In the electronic embodiment, the device 100 includes a memory storage unit which may be an internal storage unit or a removable portable memory unit. A variety of options are available for the removable portable memory storage unit: a hard disk drive and CD ROM. Small memory devices such as smart cards, electronic memory cards or PCM CIA cards (personal computer memory card industry association) may also be used to store the data. In some embodiments, the device 100 may be battery operated. A battery (not shown) may be included to provide the power source for the various components, which can be turned on or off by operating appropriate switches or activation buttons or touch or voice-activated screens. The battery may be configured as a rechargeable battery that can be recharged. In other embodiments, the device 100 may comprise a power source such as, for example, an electrical socket.

The exemplary embodiment of the controller 184 shown in FIG. 5 comprises a central processing unit (CPU) 186, a memory 188, a circuit portion 192, and an input/output interface 190. The controller 184 may be a general-purpose computer, a microprocessor, a microcontroller, or any other suitable type of computer or controller. The CPU 186 performs the processing and arithmetic operations to control the operation of the device. The memory 188 stores the computer programs, operands, and other parameters that can be used during the operation. The bus, not shown, provides for digital information transmissions between CPU 186, circuit portion 192, memory 188, and I/O 190. The bus also connects I/O 190 to portion of the device 100 that either receive digital information from, or transmit digital information to, the controller 184. I/O 190 provides an interface to control the transmission of digital information between each of the components in the controller 184. I/O also provides an interface between the components of the controller 184 and different portions of the device 100. Circuit portion 192 comprises all of the other user interface devices, such as display and keyboard, system devices, and other accessories associated with the controller 184.

A network interface may permit a connection to any network capable of being connected to an input and/or output module located within at least one or more of the following exemplary networks: an Ethernet network, an Internet Protocol network, a telephone network, a radio network, a cellular network, or any mechanism for permitting communication between two or more modes or remote locations. The connection may be implemented as a wired or wireless connection using conventional communication protocols and/or data port interfaces. In general, connection can be any communication channel that allows transmission of data between the components of the device.

In some embodiments, the network interface might also include a user interface. The user interface may include at least one component such as a keyboard, key pad, mouse, track ball, telephone, scanner, microphone, touch screen, web cam, interactive voice response system (IVR), voice recognition system or any other suitable input mechanism for conveying information. The user interface may also include an input port connected by a wired, optical, or wireless connection for electromagnetic transmissions. In some embodiments, the user interface may include connections to other computer systems to receive the input commands and data therefrom. User interface may further include a data reading device such as a disk drive for receiving input data from and writing data to storage media such as magnetic and optical disks. The device 100 may connect to a network, which may include a public network such as the Internet or a telephony network, a private network, a virtual private network, or any other mechanism for enabling communication between two or more nodes or locations. The network may include one or more of wired and wireless connections. The wireless communications may include radio transmission via the airwaves, however, those of ordinary skill in the art will appreciate that various other communication techniques can be used to provide wireless transmission including infrared line of sight, cellular, microwave, satellite, blue-tooth packet radio and spread spectrum radio. Wireless data may include, but is not limited to, paging, text messaging, e-mail, Internet access and other specialized data applications specifically excluding or including voice transmission.

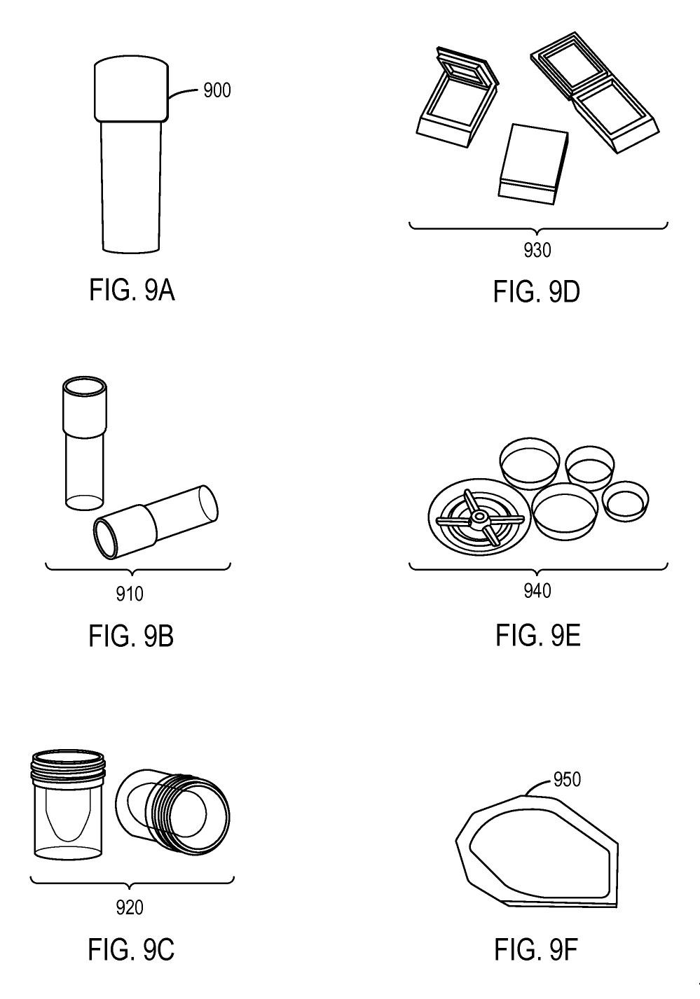

In some embodiments, one or more scales 128 (FIG. 5) may be provided, for example, within the upper surface of the sample collection zone 104 to provide a measurement of the extremity's weight and/or the specimen's weight. As shown in FIGS. 9E and 9F, weighing dishes 940, 950, can be placed on the scale 128 for weighing liquid and solid samples. In some embodiments, an electronic scale can be provided such that the electronic components can be used to carry out computer-controlled weight measurements.

In some embodiments as shown in FIGS. 5-8, the device 100 may include one or more retractable holders 130 to conveniently hold one or more pieces of collecting devices, equipment, and sample containers. The retractable holders 130 may be configured to hold various pieces of equipment such as, for example, a knife, scalpel, scissors or sample collector devices s to facilitate the collection and preparation of the specimen by the laboratory professional. These sample collectors devices are generally known as analyzer consumables. Some examples of analyzer consumables that may be employed with the device of the present teachings are shown in FIGS. 9A-9D as cuvettes 910, sample cups 920, transport/sample tubes 900, and cassettes 930. The retractable holder 130 can be positioned within and along side walls 114, 116, front wall 110, or back wall 112 of the holding member 102. Activation of the retracted holders 130 can be manually operated by a push-activated switch or electronically activated. When needed for use to hold a piece of equipment, a switch (not shown) can be activated to position the retractable holders 130 into an extended position 158. When not in use, the retractable holders 130 can be hidden within the device 100. The switch can be once again activated to retract the retractable holders 130 to a retracted position 160 within the internal housing of the holding member 102.

As illustrated in FIGS. 5 and 6, in an exemplary embodiment, the holding member 102 may include a retractable holder 130 including a single container holder. It will be understood that the holding member 102 may comprise any suitable component mounted to the interior therein for mounting the container holder 130. For example, the interior surface of the holding member 102 can include a pair of rails (not shown) disposed longitudinally into which the holder 130 is slidably mounted. Those skilled in the art would recognize a variety of techniques and devices that may be used to move the container holder 130 or container tray 178 between a retracted storage position and an extended position. In FIG. 6, the container holder 130 comprises a pair of opposed arms 152, 154 that define a container receiving area shown generally at 156. The recess and the arms define a container-receiving area 156 of varying size. The first and second opposed arms 152, 154 can be pivotably mounted by a pin and ratchet-mechanism within the holding member 102 such that when the container holder 130 is extended outward from the holding member 102, the opposed arms 152 and 154 will spring out to the outermost position for receipt of a container of various sizes, as shown in FIGS. 9A-9C.

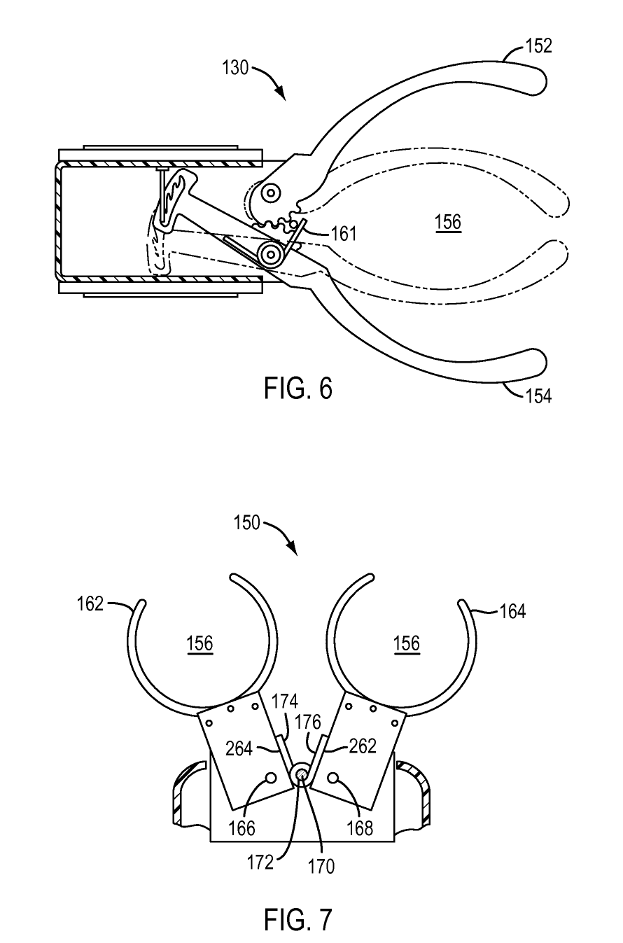

In FIG. 7, the container holder 150 comprises two holders 162 and 164 which are pivotally connected by pins 166, 168 therein. A central pin 170 can be disposed between holder 162 and holder 164. An arm spring 172 having a coil and a pair of outwardly-biased radially-extending legs 174, 176 are fixedly mounted to the central pin 170 such that the legs of the arm spring 172 bias the holders 162 and 164 radially outwardly about the pins 166, 168.

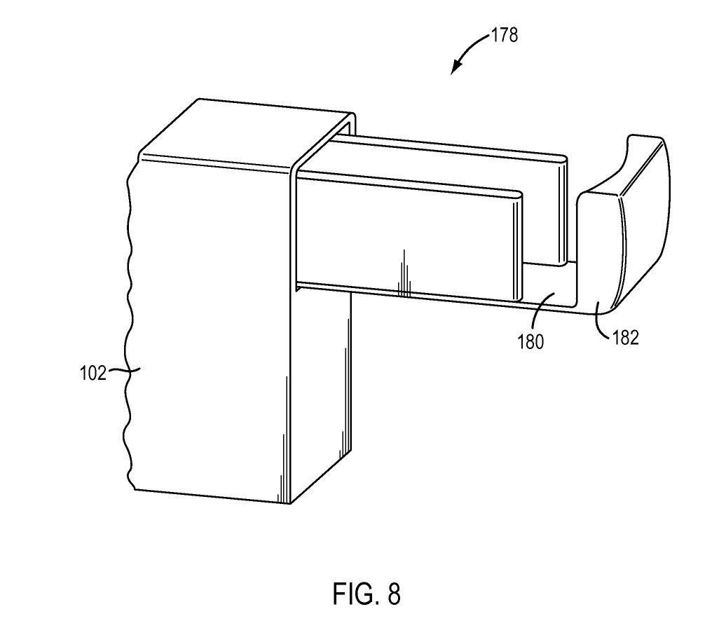

In some embodiments, in lieu of or in conjunction with a circular shaped container holder 130, 150, the container holder may be designed as a tray holder 178, as depicted in FIG. 8. The tray holder 178 may comprise a flat platform 180 that extends outwardly from the housing of the holding member 102 and further has a flange 182 extending upwardly from a forward end of the platform 180 like a drawer for receiving an analyzer consumable, such as a cassette 930. The tray holder 178 may form a drawer-like assembly which is matingly slidably inserted into the interior of the holding member 102 into a retracted position 160. It should be understood that the container holders 130, 150 and tray holders 178 shown and described in FIGS. 5-8 are nonlimiting and exemplary only. Those skilled in the art would understand that various sizes, shapes, and configurations may be envisioned for the holders without departing from the scope of the present teachings. Moreover, those having ordinary skill in the art would appreciate that the device can be designed having a variety of structures including different configurations and number of holders.

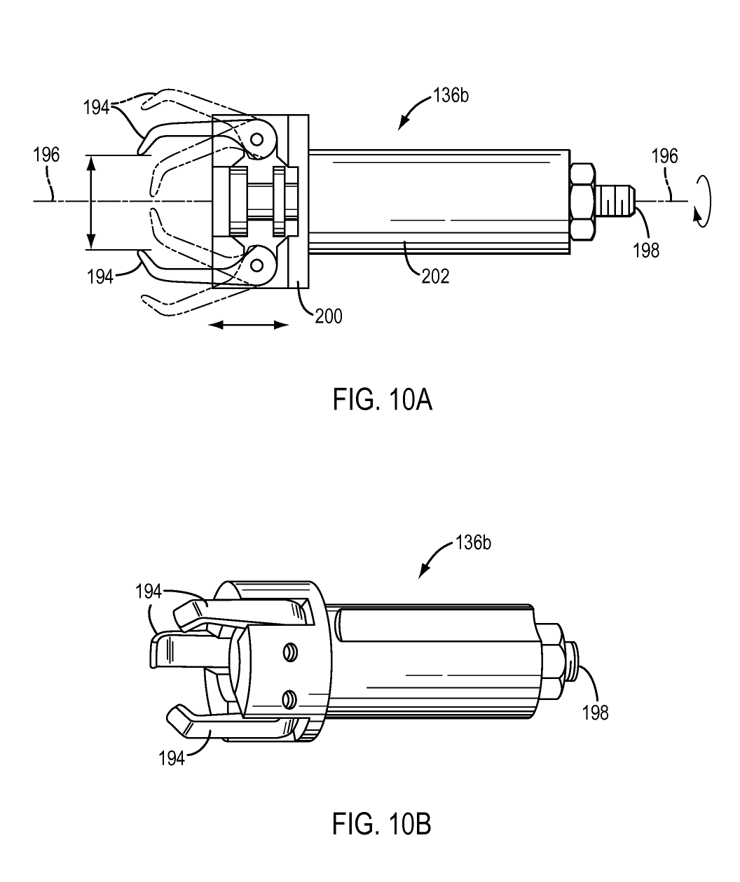

As shown in FIGS. 5, 10A, 10B and 11, the device 100 can include a pair of grippers 136 that holds and rotates the amputated extremity 204 (FIG. 11) during the sample collection process. The various structures and operations of automated or powered grippers to grip or retain work pieces are well known. Therefore, without entering into details, which are not relevant to the description of the present teaching, well-known structures and functions of the gripper will not be described in detail. FIGS. 10A and 10B depict the structure of a general gripper that can be employed within the device 100. In general, the gripper is a relatively compact device of simple construction for handling an amputated extremity 204 with relatively gentleness to avoid causing damage to the extremity. The gripper can be configured, for example, as a clamp 136a or claw 136b. The clamp 136a can be configured with an adjustable inflatable interior pad or bladder 137 that is capable of adjusting to different size extremities. A pressure source may also be provided for inflating the interior pad or the bladder. The claw 136b configuration may be defined by two or more fingers 194, as shown in FIGS. 10A and 10B. During insertion of the extremity, each clamp 136a or claw 136b can be adjusted and rotated independently of the other so that the extremity can be securely placed therein. Each clamp 136a or claw 136b can be independently adjusted to handle amputated extremities of different sizes and the awkward shape of the extremity.

FIG. 10A illustrates an embodiment of the gripper 136b having two opposing fingers being adjusted between a fully open position to a closed or gripping position. The claw 136b can be pivotally mounted within each holding member 102 by a pin 198 such that it rotates about axis 196. During operation with the amputated extremity 204 retained therein (FIG. 11), both grippers (clamps 136a or claws 136b) can rotate in synchronization with each other, similar to a lathe. To insure that the grippers 136a, 136b will not inadvertently release the extremity during rotation, the inflatable bladder 137 or fingers 194 may be set in a locked position to grip and retain the extremity. In some embodiment, the head portion 200 of the gripper 136a, 136b may be configured to extend outwardly from within the body portion 202 and retracted therein. FIG. 10B illustrates an embodiment of the claw 136b having three fingers 194.

A barcode printer (not shown) may be provided within the interior housing of the holding member 102 for imprinting patient identifying information in the form of a barcode 210 (FIG. 11). The barcode printer can be constructed based on well-known portable barcode and label printer technology, such as those described in U.S. Pat. Nos. 5,092,688 and 4,145,966, the contents of which are incorporated by reference herein. During the sample collection process, the specimen of the amputated extremity 204 and the analyzer consumables 900, 910, 920 and 930 can be barcoded such that the identifying information matches to insure proper patient identification when the items are retrieved and transported to another location for further analysis. Furthermore, the electronic components of the device can interface with a network system that contains, for example, patient pathology information to automatically record and document the specimen parameters without requiring medical transcription. Another benefit of the automated features of the device is that once the barcodes are applied, the specimen and samples can be automatically and continuously monitored and tracked by the network's computer system to insure positive patient identification and location. In some embodiments, the device may also include an audio tape recorder to record patient information. The device may also automatically download the audio recordings to a form of portable or permanent storage media.

Collecting and preparing a sample typically involves numerous steps. The device 100 according to the present teachings can be used to collect and prepare a sample of an extremity amputated, for example, for soft tissue tumor. In use, the amputated extremity may be inserted into the extremity holder 102 and secured into position by straps 122 (FIG. 1) or in an alternate embodiment secured within grippers 136a (FIG. 5) or 136b (FIGS. 10A and 10B). The adjusting rods 106 may be longitudinally extended to accommodate the extremity. Using the ruler 126 and other measuring instruments, measurements of the length and the circumference of the extremity are taken, including a measure of the circumference at the level of the tumor.

In an electronic embodiment, the measurements and other pertinent analysis and information can be automatically recorded and stored in real-time within the mini-computer 186 of the device 100 or on a portable small memory device removably inserted within the device. The measurements and information may also be directly or wireless transmitted to another computer or network. A determination of the presence, position, and dimension of biopsy sites can be made. Then, the laboratory professional searches for the major lymph node groups, identifies and places them in one or more of the containers 900, 910, 920 and 930 held within the retractable holders 130. Using a knife or scalpel which may also be conveniently held by a retractable holder 130, cuts through the skin are performed to carefully dissect the subcutaneous fat, muscles, and major arteries, veins and nerves around the tumor, while avoiding cutting through the tumor. Next, the laboratory professional tries to determine as accurately as possible the relationship of the tumor with the following structures: skin, subcutaneous fat, specific muscles, arteries, veins and nerves, periosteum, and bone. As soon as all the margins of the tumor have been determined, the entire area with a good margin of normal tissue can be removed using a scalpel and scissors. In order to study the specimen thus obtained, one common practice is to divide the tumor slices with a sharp knife. The laboratory professional continues the dissection with the forceps, scissors, and scalpel to determine the tumor relationship with the structures previously mentioned. To take a sample for histology, several pieces from different areas of the mentioned structures are placed in a container of formalin held by a retractable holder 130. These specimens may be fixed for a predetermined time in formalin container, and then trimmed to place be in cassettes 930, which can also be held by the retractable tray holders 178. Then, the laboratory professional dissects the soft tissues from the rest of the extremity, looking for other foci of tumor or other lesions. The major bone of the extremity may be cut longitudinally using, for example, a saw, cutting one of the sections through the area of bone closest to the soft tissue tumor. A further examination for tumor extension or other lesions can be made. Next, the major joints can be opened and examined until the laboratory professional completes the sample collection and preparation process.

### DEFINITIONS

In describing the invention, the following definitions are applicable throughout the description provided above).

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, or a chip set; a system on a chip (SoC), or a multiprocessor system-on-chip (MPSoC); an optical computer; a quantum computer; a biological computer; and an apparatus that may accept data, may process data in accordance with one or more stored software programs, may generate results, and typically may include input, output, storage, arithmetic, logic, and control units.

“Software” may refer to prescribed rules to operate a computer. Examples of software may include: software; code segments; instructions; applets; pre-compiled code; compiled code; interpreted code; computer programs; and programmed logic.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash removable memory; a memory chip; and/or other types of media that may store machine-readable instructions thereon.

A “computer system” may refer to a system having one or more computers, where each computer may include a computer-readable medium embodying software to operate the computer. Examples of a computer system may include: a distributed computer system for processing information via computer systems linked by a network; two or more computer systems connected together via a network for transmitting and/or receiving information between the computer systems; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet. Exemplary networks may operate with any of a number of protocols, such as Internet protocol (IP), asynchronous transfer mode (ATM), and/or synchronous optical network (SONET), user datagram protocol (UDP), IEEE 802.x, etc.

It will be apparent to those skilled in the art that various modifications and variations can be made to the amputated extremity holder and sample collection device of the present disclosure without departing from the scope its teachings. Other embodiments of the disclosure will be apparent to those skilled in the art from consideration of the specification and practice of the teachings disclosed herein. It is intended that the specification and examples be considered as exemplary only.

What is claimed is:

1. A portable amputated extremity holder and sample collection device comprising:
a first supporting member;
a second supporting member;
a pair of rods disposed so as to adjustably connect the first supporting member and the second supporting member to form an extremity holder and a sample collection zone;
the extremity holder is configured to hold an amputated extremity during an examination for performing a sample collection procedure on the amputated extremity; and
the sample collection zone comprises a plurality of testing and measuring components associated with at least one of the supporting members to assist with the sample collection procedure.

2. The device of claim 1, wherein each of the pair of rods comprise links such that at least one of the adjusting rods is adjustable relative to the other adjustable rod to provide a spacing between the first and second supporting member to accommodate amputated extremities of various lengths.

3. The device of claim 1, wherein the pair of rods is movable from an extended position to a collapsed and folded position to provide easy storage and transport.

4. A portable amputated extremity holder and sample collection device comprising:
a first supporting member;
a second supporting member;
a pair of rods disposed so as to adjustably connect the first supporting member and the second supporting member to form an extremity holder and a sample collection zone;
the extremity holder is configured to hold an amputated extremity during an examination for performing a sample collection procedure on the amputated extremity;
the extremity holder comprises a pair of opposed rotating holding members for securely supporting the amputated extremity without damaging the amputated extremity and configured to rotate the amputated extremity during the sample collection procedure; and
the sample collection zone comprises a plurality of testing and measuring components associated with at least one of the supporting members to assist with the sample collection procedure.

5. The device of claim 4, wherein the extremity holder further comprises a rotating mechanism for rotating and adjusting the opposed rotating holding members individually or collectively in order to insert independently and securely retain a first end and a second end of the amputated extremity at different angular positions.

6. The device of claim 5, wherein the opposed rotating holding members defines a clamp configuration having an adjustable inflatable pad or an adjustable inflatable bladder capable of adjusting to securely hold the amputated extremity.

7. The device of claim 5, wherein the opposed rotating holding member defines a claw configuration having a plurality of fingers capable of adjusting to securely hold the amputated extremity.

8. A portable amputated extremity holder and sample collection device comprising:
a first supporting member;
a second supporting member;
a pair of rods disposed so as to adjustably connect the first supporting member and the second supporting member to form an extremity holder and a sample collection zone;
the extremity holder is configured to hold an amputated extremity during an examination for performing a sample collection procedure on the amputated extremity;
the sample collection zone comprises a plurality of testing and measuring components associated with at least one of the supporting members to assist with the sample collection procedure; and
the sample collection zones comprises at least one retractable component movable from a retracted position within a housing of at least one of the supporting member to an extended position in which the at least one retractable holder extends from the housing of the at least one of the supporting member.

9. The device of claim 8, wherein the plurality of testing and measuring components comprises a ruler to measure dimensions of the amputated extremity.

10. The device of claim 9, wherein the ruler comprises a retractable ruler movable from the retracted position such that the retractable ruler partially retracts into the housing of the at least one supporting member to the extended position.

11. The device of claim 8, wherein the plurality of testing and measuring components comprises at least one scale for receiving and weighing at least one of:
the amputated extremity;
a specimen of the amputated extremity; and
a sample obtained from the amputated extremity.

12. The device of claim 11, wherein the at least one scale is configured to receive a weighing tray to sit on a weighing surface of the scale for weighing the sample.

13. The device of claim 12, wherein the at least one scale comprises an electronic scale.

14. The device of claim 8, further comprising at least one retractable holder comprising a collapsible structure which can fold to fit inside the housing of the at least one supporting member and is slidable into and out of the housing.

15. The device of claim 14, wherein the at least one retractable holder comprises at least one single holder having a pair of opposed adjustable arms that define a container receiving area to holder containers of varying sizes.

16. The device of claim 14, wherein the at least one retractable holder comprises a tray holder configured to define a drawer capable of extending outwardly from the housing of the at least one support member and retracting within the housing.

17. The device of claim 14, wherein the at least one retractable holder is configured to hold at least one analyzer consumable selected from at least one of:
a cuvette,
a sample cup,
a transport tube,
a sample tube, and
a cassette.

18. The device of claim 8, further comprising a barcode generator for imprinting identifying information in the form of a barcode to be applied to at least one of the amputated extremity, a specimen, and a sample obtained from the amputated extremity.

19. A portable amputated extremity holder and sample collection device comprising:
a first supporting member;
a second supporting member;
a pair of rods disposed so as to adjustably connect the first supporting member and the second supporting member to form an extremity holder and a sample collection zone;
the extremity holder is configured to hold an amputated extremity during the examination;
the sample collection zone comprises a plurality of testing and measuring components associated with at least one of the supporting members to assist with the sample collection procedure;
a processor provided within the device to communicate with and coordinate operation of various sensors and components to control the device;
the processor connected to a network;
the network connects and interfaces with a patient pathology information system to generate, report, store and transmit patient information related to the amputated extremity;
the extremity holder comprises a pair of opposed rotating holding members for securely supporting the amputated extremity without damaging the amputated extremity and configured to rotate the amputated extremity during the sample collection procedure;
the sample collection zones comprises at least one retractable component movable from a retracted position within a housing of at least one of the supporting member to an extended position in which the at least one retractable holder extends from the housing of the at least one of the supporting member;
the processor controls:
movement of the pair of rods to adjustably connect the first and second holding members,
movement of the pair of opposed rotating holding members, and
movement of the at least one retractable component; and
a display in communication with the processor and configured to display the patient information.

20. The device of claim 19, wherein the network interface transmits digital information from the processor to a remote location over a communication network.

21. The device of claim 20, wherein the network includes one or more wired and wireless connections.

22. The device of claim 21, wherein each of the pair of rods comprise links such that at least one of the adjusting rods is adjustable relative to the other adjustable rod to provide a spacing between the first and second supporting member to accommodate amputated extremities of various lengths.

23. The device of claim 21, wherein the extremity holder further comprises a rotating mechanism for rotating and adjusting the opposed rotating holding members individually or collectively in order to insert independently and securely retain a first end and a second end of the amputated extremity at different angular positions.

24. The device of claim 21, wherein the at least one retractable holder comprises at least one of:
a. at least one single holder having a pair of opposed adjustable arms that define a container receiving area to holder containers of varying sizes, and
b. a tray holder configured to define a drawer capable of extending outwardly from the housing of the at least one support member and retracting within the housing.

25. The device of claim 24, wherein the at least one retractable holder is configured to hold at least one analyzer consumable selected from at least one of:
a cuvette,
a sample cup,
a transport tube,
a sample tube, and
a cassette.

26. The device of claim 21, wherein the device further comprises at least one of:
a. an electronic retractable ruler
b. an electronic scale, and
c. an electronic barcode generator.

27. The device of claim 26, wherein the electronic barcode generator imprints identifying information in the form of a barcode to be applied to at least one of the amputated extremity, a specimen, and a sample obtained from the amputated extremity.

28. The device of claim 27, wherein after the barcode is applied, the network automatically and continuously monitors and tracks the at least one of the amputated extremity, a specimen, and a sample obtained from the amputated extremity to insure positive patient identification and location.
